(19) United States
(12) Patent Application Publication
(10) Pub. No.: US 2008/0021265 A1
(43) Pub. Date: Jan. 24, 2008

(54) Prosthesis to be implanted in a human body for repairing or treating pelvic organ prolapses in a female patient and surgical method using such a prosthesis

(76) Inventors: Olivier Garbin, Strasbourg (FR); Arnaud Wattiez, Strasbourg (FR); Michel Cosson, Lambersart (FR)

Correspondence Address: YOUNG & THOMPSON, 745 SOUTH 23RD STREET, 2ND FLOOR, ARLINGTON, VA, 22202, US

(21) Appl. No.: 11/806,314

(22) Filed: May 31, 2007

Related U.S. Application Data
(60) Provisional application No. 60/809,366, filed on May 31, 2006.

(51) Int. Cl. A61F 2/02

(52) U.S. Cl. 600/30

(57) ABSTRACT

A procedure for treating pelvic organ prolapses in a female patient using a synthetic, biological or mixed implantable prosthesis includes steps of: a) preparing the patient as for a conventional laparoscopy; b) performing rectovaginal dissection down to the levator-ani; c) dissecting the vesico-vaginal space, preferably up to the bladder trigone; d) providing a two part prosthesis including an anterior part and a posterior part, each having a central body and two arm like strap portions; e) placing the posterior part into the rectovaginal space and pressing through and anchoring posterior strap portions into the right and left puborectal muscles; f) placing anterior part into the vesico-vaginal space; g) passing the anterior strap portions, once or twice, through holes of the posterior part and attaching to the uterosacral ligaments; h) adjusting concerned organs by applying tension to strap portions; i) closing the patient.

The present invention relates to a surgical procedure for repairing pelvic organ prolapses with a synthetic or biological prosthesis.

Gynecare (Somerville, N.J., USA) recently launched on the market a product named PROLIFT®. The surgical procedure which is used in association to this product, consists in performing, through a vaginal approach, an anatomical repair of both the anterior and posterior pelvic fasciae with the use of a synthetic mesh.

A description of a prosthesis of that known type and of the surgical procedure associated with is disclosed in U.S. Pat. No. 7,131,944, the content of which is integrally enclosed herein by reference.

A popular surgical alternative, named sacro-colpo-suspension or sacro-colpo-pexy consists in performing a suspension of the genital apparatus to the sacral promontory. As for PROLIFT, this procedure requires the use of a synthetic or biological prostheses. The procedure in its more advanced version is performed laparoscopically. While the clinical outcomes of laparoscopic suspension are good, the procedure has not gained wide acceptance because of its surgical difficulty. Attempts have been made in order to make lap sacro-colpo-pexies simpler but without much success.

The inventors believe that instead of trying to simplify the lap sacro-colpo-pexy, it would be wiser to abandon the concept of suspension to the sacral promontory and to try to replicate laparoscopically a repair, the outcome of which is similar to the one made by vaginal approach by the procedure associated with the PROLIFT product.

Therefore, the purpose of this invention is to describe a surgical procedure for providing the same or closely similar repair as the one provided by PROLIFT, the repair being made laparoscopically instead of through a vaginal approach.

According to a first aspect of the invention, there is provided a prosthesis to be implanted in a body for repairing or treating pelvic organ prolapses in a female patient, said one piece prosthesis being made of a synthetic, biological or mixed mesh like fabric, said prosthesis having roughly a human shape like outline with two arm portions, two leg portions, a trunc portion and a head portion, the head and trunc portions forming together substantially a rectangular shape divided into an interior part and a posterior part by a hypothetical medium line crossing the trunc portion under its junctions with the arm portions; the posterior part being provided with two separated holes situated near the medium line, the anterior part being adapted to be placed into the vesico-vaginal space and attached low in said space, the posterior part being adapted to be placed into the rectovaginal space, the leg portions being adapted to be passed through and anchored into the right and left puborectal muscles and the arm portions being adapted to be passed through the holes of the posterior part and embedded within and/or attached to the uterosacral ligaments.

Alternatively, depending on the prolapse to be treated, the posterior part can also be without any leg portions, said part being left free or suspended to the muscle in the recto-vaginal space by an adapted means (sutures, glue, tacker, staples, . . . ).

In accordance with an other embodiment of said first aspect of the invention, there is made provision for a prosthesis to be implanted in the body of a female patient for repairing or treating pelvic organ prolapses, said prosthesis being formed of two parts made of synthetic, biological or mixed mesh like fabric,

wherein a first or anterior part comprises a main rectangular shaped body provided with two arm like anterior strap portions extending with a diverging angle from the corners adjacent a small side of the main body, said main body having a truncated shape at its other small side and a hole near the edge of the small side between the junctions with the arms, wherein a second or posterior part comprises a main rectangular shaped body provided with two arm like posterior strap portions extending with a diverging angle and a slightly curved shape from the corners adjacent a small side of the main body, said main body having two separated holes next to its other opposite small side, the main body of the anterior part being adapted to be placed into the vesico-vaginal space and attached low in said space, with the corresponding anterior strap portions adapted to be passed, once or twice, through the holes of the posterior part and attached to the uterosacral ligaments, the main body of the posterior part being adapted to be placed into the rectovaginal space, with the corresponding posterior strap portions adapted to pass through and being anchored into the right and left puborectal muscles.

Preferably, the mesh fabric consists in a bio-compatible advantageously knitted polypropylene material fabric and in that the dimensions of the prosthesis constituting parts or portions are adapted to the size of the patient's anatomy (different sizes and shapes of the mesh fabric are possible).

According to a second aspect of the invention, a procedure is provided for repairing or treating pelvic organ prolapses in a female patient using a synthetic, biological or mixed implantable prosthesis comprising the steps of:

a) setting-up the patient as for a conventional laparoscopy;

b) performing a rectovaginal dissection down to the levator-ani;

c) performing a dissection of the vesico-vaginal space, preferably up to the bladder trigone;

d) providing a prosthesis made of mesh like fabric and formed by central anterior and posterior portions and lateral arm and possible leg portions and inserting said prosthesis through the dissected area so as to place the posterior part into the rectovaginal space;

e) anchoring the posterior leg portions into the perineum or leaving the posterior part free in the recto-vaginal space;

f) folding down the anterior part into the dissected vesico-vaginal space;

g) passing the anterior arm portions through the holes of the posterior part and anchoring said arm portions into the uterosacral ligaments or passing said anterior arm portions under the peritoneum or along the uterosacral ligament in order to restore a physiological suspension of the uterus, the cervix or the vaginal apex in case of post hysterectomy prolapse;

h) adjusting the position of the concerned organs by applying tension to the arm and leg portions, the suspension being achieved by the friction of the mesh in the native tissues;

i) closing up the patient.

Advantageously, the prosthesis used is a prosthesis according to the first alternative.

Preferably, a subtotal hysterectomy is performed after step c) and the uterine cervix is closed and a needle suspension to the anterior abdominal wall is performed after step d).

In accordance with an other embodiment of said second aspect of the invention, a procedure is provided for repairing or treating pelvic organ prolapses in a female patient using a synthetic, biological or mixed implantable prosthesis comprising the steps of:

a) setting-up the patient as for a conventional laparoscopy;

b) performing a rectovaginal dissection down to the levator-ani;

c) performing a dissection of the vesico-vaginal space, preferably up to the bladder trigone;

d) providing a two part prosthesis comprised of an anterior part having a central body and two arm like strap portions and a posterior part having a central body and two arm like strap portions;

e) introducing and placing the posterior part into the rectovaginal space and pressing through and anchoring the posterior strap portions into the right and left puborectal muscles;

f) introducing and placing the anterior part into the vesico-vaginal space;

g) passing the anterior strap portions, once or twice, through the holes of the posterior part and attaching them to the uterosacral ligaments;

h) adjusting the position of the concerned organs by applying tension to the strap portions;

i) closing up the patient.

Advantageously, the prosthesis is a prosthesis as described before as a second alternative.

Preferably, a subtotal hysterectomy is performed after step c) and a suture is passed to close the cervix, said suture passing also through an inbuild hole of the anterior part.

According to a feature of the invention, the passing, placing and attaching of the arm and/or leg portions of the prosthesis is performed by grasping and then pulling said portions by their respective free ends by means of an introducer comprising a specifically shaped needle (see figure) cooperating with a corresponding canula.

Furthermore, one or several of the following features can be provided or performed in connection with the invention:
the anchoring of the posterior leg or strap portions is performed by passing the introducer through the puborectal muscles within two lateral incisions made in the perineum;
the attachment of the anterior arm portions is performed by passing the introducer through incisions of the perineum and basting the needle under the perineum until the root of the uterosacral ligament is transfixed, before grasping the free end of the concerned anterior arm portion;
the attachment of the anterior strap portions to the uterosacral ligament is performed by passing the introducer successively through the following tissues: recto-vaginal peritoneum, uterosacral-cardinal complex, possibly the vesico-uterine fascia, in and out the vesico-uterine peritoneum, before grasping the free end of the concerned anterior strap portion;
the free edge of the anterior part of the prosthesis is attached low in the vesico-vaginal space.

As an alternative, the posterior part of the prosthesis can be without leg portions or posterior strap portions, the posterior part of the prosthesis being free or suspended to the muscle by an adapted means.

Thus, according to the invention, a prosthesis made of mesh like fabric is placed under the peritoneum to restore a normal anatomy and to make the suspension, in particular along the cervical ring and the utero-sacral ligaments. This placement is made without tension (“tension-free”), preferably with a needle covered by a canula. After each removal of the needle, the corresponding arm or leg of the mesh is passed through a corresponding canula to avoid any injury to the surrounding tissu. Then, after adjustment of the tension, the concerned canula is removed, and the prosthesis is left in place, under the peritoneum.

The invention will be better understood thanks to the following description and drawings of different embodiments of said invention given as non limitative examples thereof, wherein:

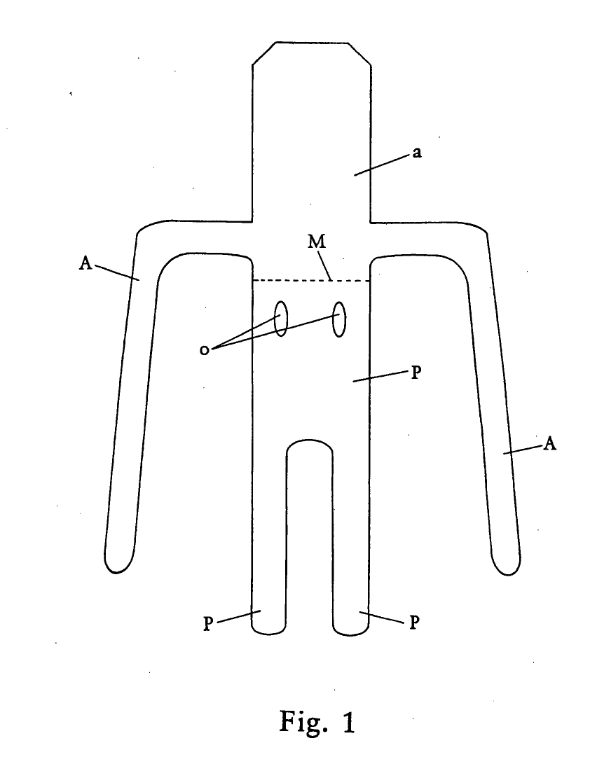

FIG. 1 is a plan view of a one piece prosthesis according to a first aspect of the invention;

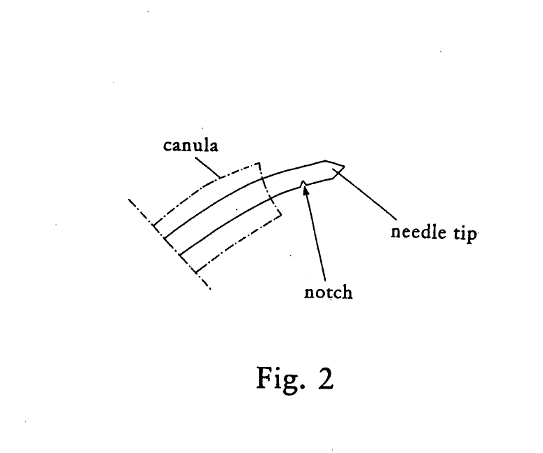

FIG. 2 is a detail view of the tip of an introducer used in connection with the prosthesis of FIG. 1, the needle having a notch within its internal curvature;

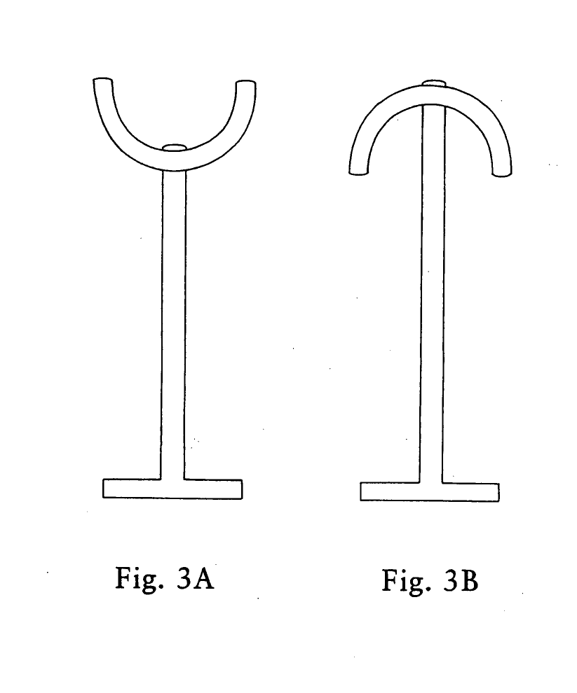

FIGS. 3A and 3B are lateral views of a uterine cervix pusher used in connection with the prosthesis of FIG. 1 and provided with a reversable cup, in order to facilitate the surgical handling of the uterus;

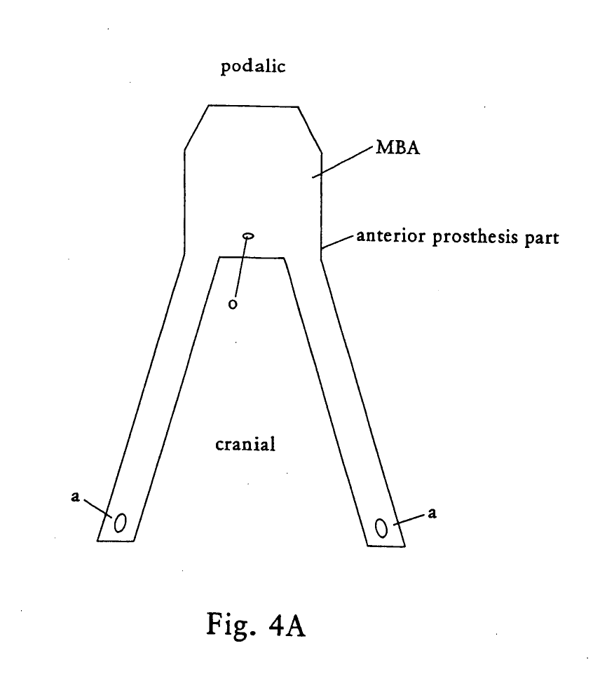

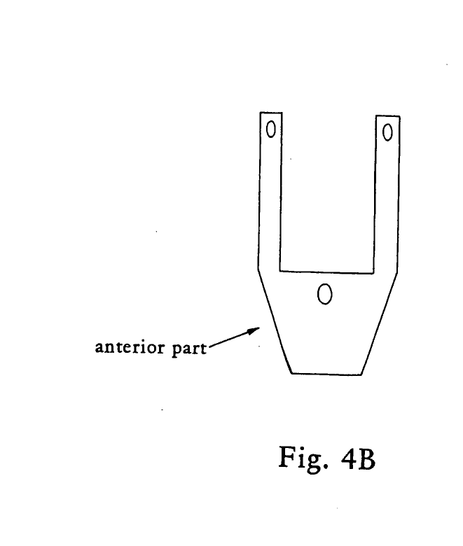

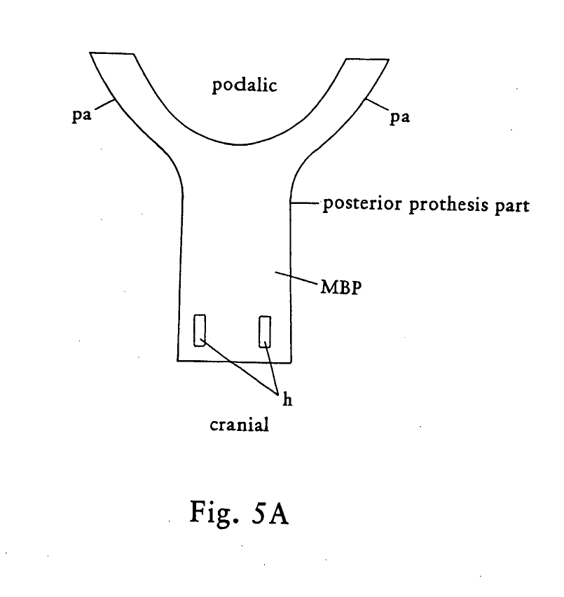

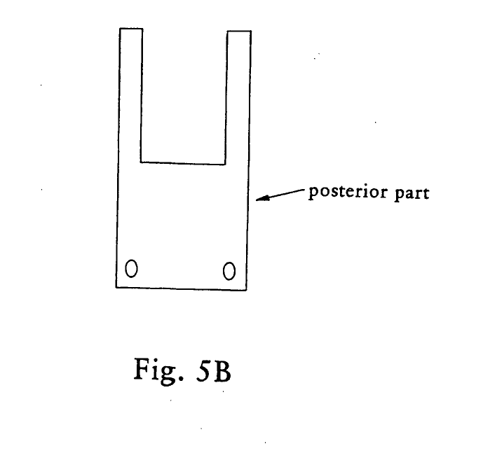

FIG. 4A and FIG. 5A are plan top views of the two parts of a prosthesis according to a first alternative of an other embodiment of the invention, to be used in connection with the second embodiment of the surgical procedure of the invention;

FIGS. 4B and 5B are views similar to the FIGS. 4A and 5A of a second alternative of an other embodiment of the invention, also to be used in connection with the second embodiment of the surgical procedure of the invention;

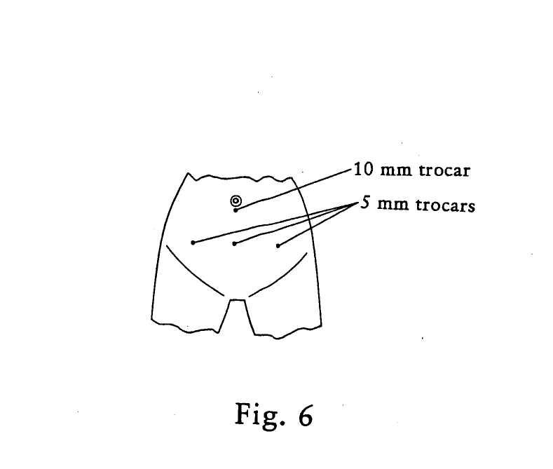

FIG. 6 is a partial top view of the abdomen of a female body illustrating the implantation sites of the trocar devices;

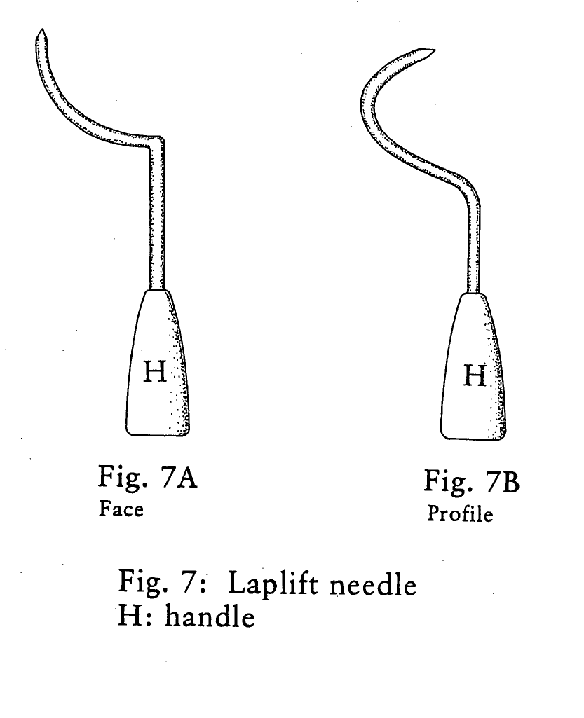

FIGS. 7A and 7B are respectively frontal and lateral plan views of a specifically designed helicofdal needle to be used in connection with the second embodiment of the surgical procedure of the invention;

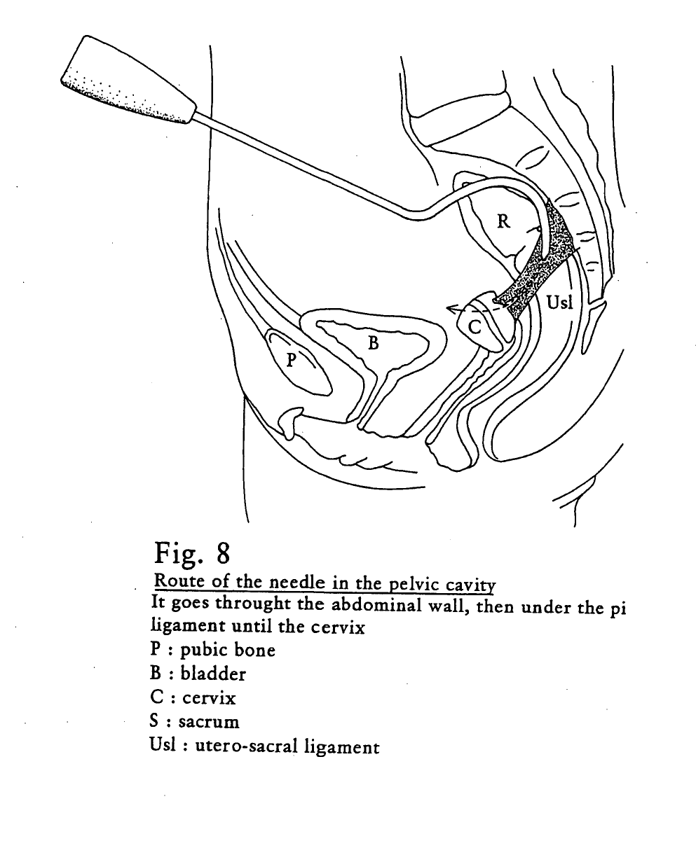

FIG. 8 is a shematical sectional view illustrating the route of the needle in the pelvic cavity during the surgical procedure of the invention (second embodiment);

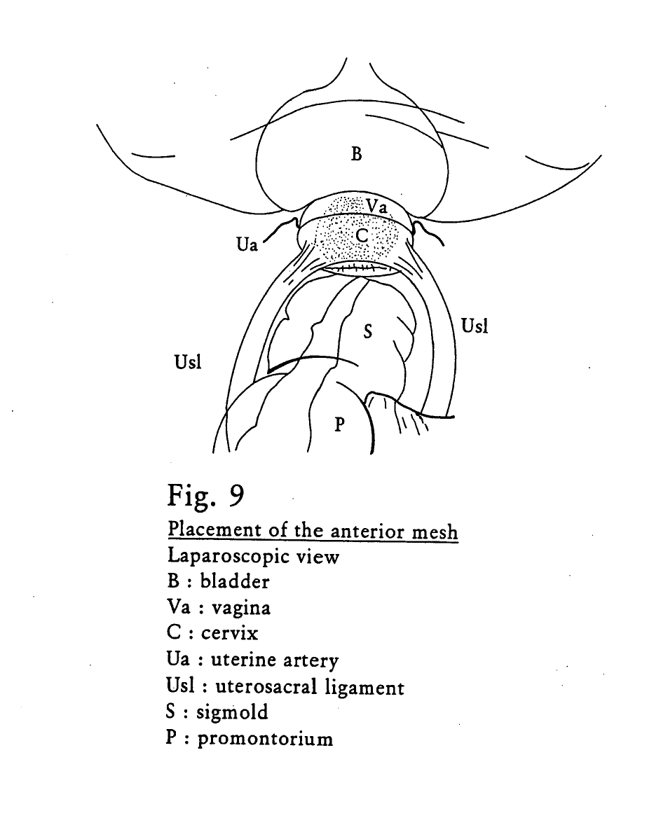

FIG. 9 is a shematical laparoscopic view showing the placement of the anterior part of the prosthesis;

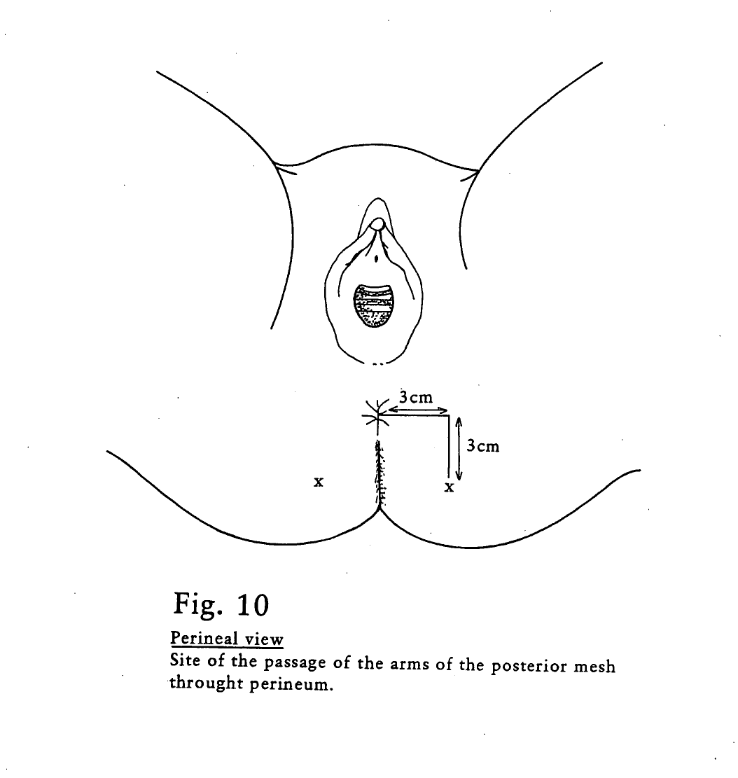

FIG. 10 is a partial shematical perineal view showing the sites of the passage of the posterior arms of the posterior part of the prosthesis through the perineum;

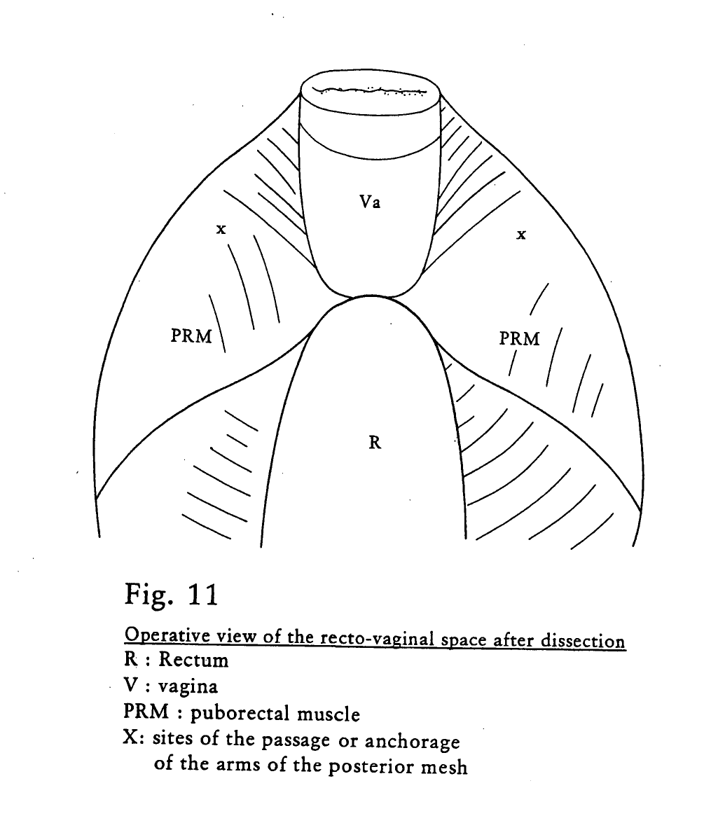

FIG. 11 is a shematical operative view of the recto-vaginal space after dissection before the posterior part of the prosthesis is part in place;

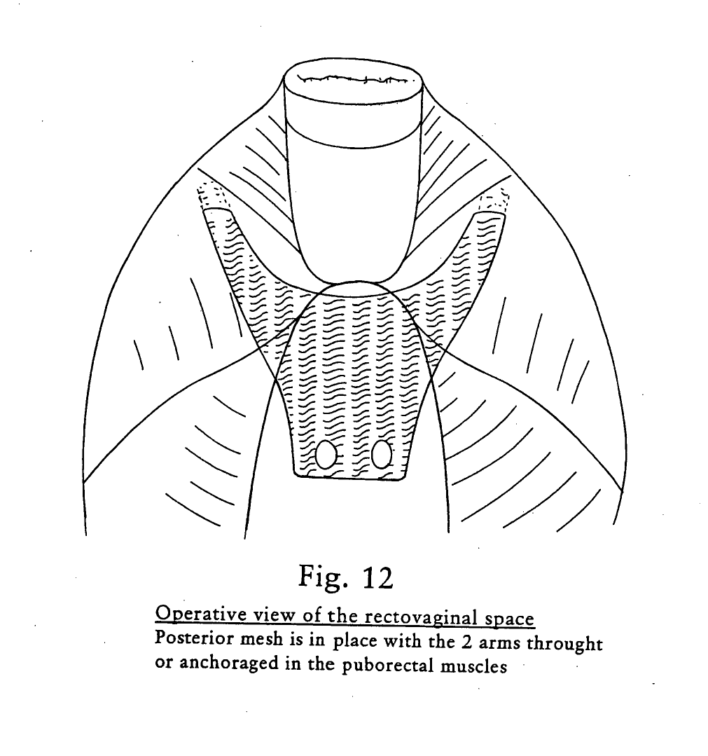

FIG. 12 is a shematical operative view of the recto-vaginal space analog to FIG. 11, after the posterior part of the prosthesis is in place with the two posterior arms through and anchored in the puborectal muscles;

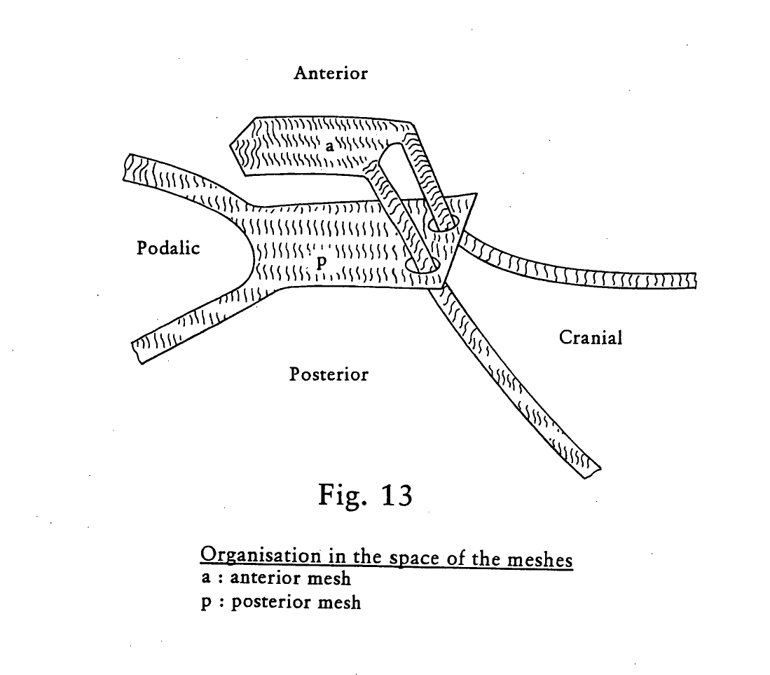

FIG. 13 is a shematical perspective view illustrating the spatial organisation and interconnection of the two parts of the prosthesis, and,

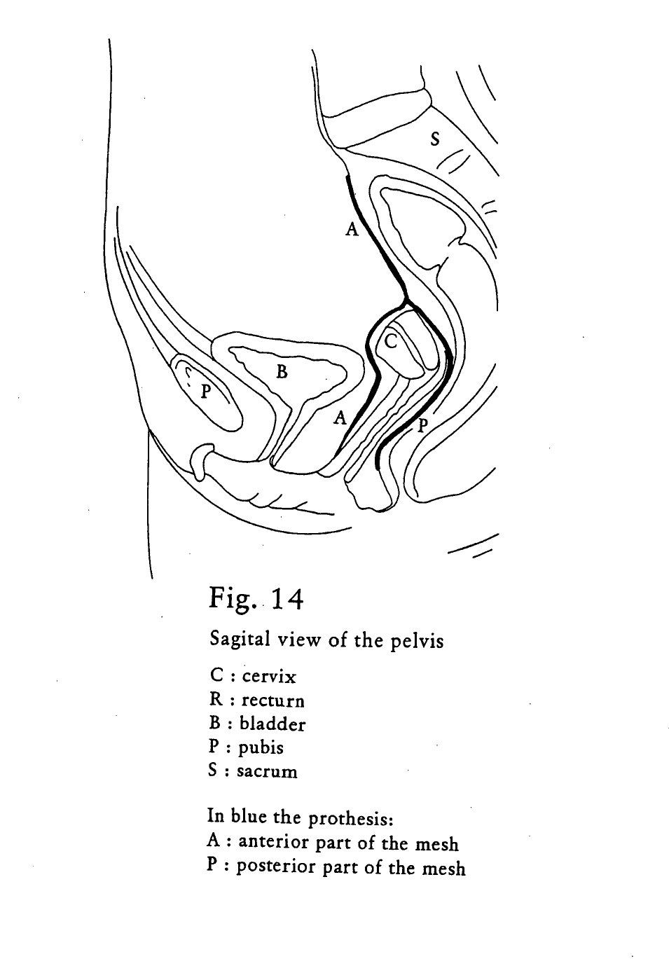

FIG. 14 is a shematical sectional sagittal view of the pelvis after both parts of the prosthesis have been put in place and interconnected.

The invention will now be described, in a non limitative manner, with reference to two embodiments, each of them comprising several alternatives.

### First Embodiment of the Invention

Preparation

The operative room is set up for a standard laparoscopic surgery. General anesthesia is required.

The patient is placed in dorsal lithotomy position with an access to the perineum which is prepared for an approach. The anus is isolated from the wound with a sterile drape.

Three 5 mm trocars or 5 mm trocars plus a 12 mm trocar are inserted suprapubically. A 10 mm trocar is placed in the ombilicus for passing a 0 or 30° angle optic system.

The procedure comprises the following steps:

Step 1:

An intrauterine manipulator is inserted. The digestive loops are kept out of the operative wound.

Step 2:

The recto-vaginal space is dissected up to the ano-rectal junction and the levator ani muscles.

Step 3:

A subtotal hysterectomy is performed when needed. The vesico-vaginal space is dissected up to the bladder trigone.

Step 4:

The uterine cervix is closed. A needle suspension to the anterior abdominal wall is performed (needle is kept).

The next steps are different depending if the uterus is conserved or not.

Alternative 1: Without uterine conservation (Subtotal hysterectomy)

Step 5.1:

The mesh forming the prosthesis as shown in FIG. 1 is inserted.

Step 6.1: Transperineal passage of the posterior legs.

A 1 cm incision is performed on the perineal skin 3 cm lateral and 3 cm below the anus on each side. Under visual control, a needle within a canula is passed from the perineal incisions through the plan of the levator ani muscles. The posterior legs (“P”) are caught in the notch of a specifically designed needle as shown in FIG. 2 and then withdrawn to the perineum through the canula on both sides.

Step 7.1:

The “a” part of prosthesis as shown in FIG. 1 is fold down in the dissected vesico-vaginal space.

Optionally, the inferior part of “a” can be fixed to the deepest aspects of the vesico-uterine dissection by a non-transfixing suture and/or the cervix can be attached to the “M” level of the prosthesis by the suture which has been used for fixation to the abdominal wall.

Step 8.1:

After identification of the ureters, the peritoneum is incised laterally to the rectum about 2 cm below the sacral promontory on both sides.

Under visual control, the needle within its canula is basted under the peritoneum until the root of the uterosacral ligament is transfixed.

Step 9.1:

Each arm (“A”) of the prosthesis is caught by the same specifically designed needle and withdrawn through a corresponding canula.

Step 10.1:

The canula are withdrawn and the peritoneum is closed. Optionally, the arms (“A”) can be attached to each other and to the peritoneal suture.

Alternative 2: With uterine conservation

Step 5.2:

The prosthesis as shown in FIG. 1 is divided in two parts, namely “anterior” part and “posterior” part, at the level of the “M” line. The posterior prosthesis is introduced within the peritoneal cavity.

Step 6.2: Transperineal passage of the posterior legs

A 1 cm incision is performed on the perineal skin 3 cm lateral and 3 cm below the anus on each side. Under visual control, a needle within a canula is passed from the perineal incisions through the plan of the levator ani muscles. The posterior legs (“P”) are caught in the notch of a specifically designed needle as shown in FIG. 2 and then withdrawn to the perineum through the canula on both sides.

Step 7.2:

The “anterior” prosthesis is introduced within the peritoneal cavity and placed in the dissected vesico-uterine space. Optionally, the inferior part of “a” can be fixed to the deepest aspects of the vesico-uterine dissection by a non-transfixing suture.

Step 8.2:

After identification of the ureters, the peritoneum is incised laterally to the rectum about 2 cm below the sacral promontory on both side.

Under visual control, the needle within its canula is basted under the peritoneum until the root of the uterosacral ligament is transfixed. Then, the needle within its canula is passed through the orifice (“o”) of the posterior prosthesis and then within the broad ligament up to the dissected vesico-vaginal space.

Step 9.2:

Each arm (“A”) of the prosthesis is caught by the same specifically designed needle and withdrawn through the canula.

Step 10.2:

The canula are withdrawn and the peritoneum is closed. Optionally, the arms (“A”) can be attached to each other and to the peritoneal suture.

The following three alternative variations of the procedure described herein before can be contemplated:

Variation 1

In this variation, the right arm (“A”) is attached by a suture to the sacral promontory.

Variation 2

In this variation, the prosthesis comprises an upper arm in its midline to be passed either across the gluteus or the obturator foramen or the sacrospinous ligament.

Variations 3

In this variation two prosthesis mesh parts are used. The posterior mesh is placed as in the standart manner described before. The anterior mesh is attached to the deepest aspects of the vesico-uterine dissection by a non-transfixing suture. The other part of the mesh is made with two arms which are passed through the lateral aspect of the cervix and then through the highest part of the posterior mesh. Then a passer introduced at the root of the US ligament passes the two arm in a tension free manner along the US ligaments.

### Second Embodiment of the Invention

In this second embodiment of the invention, a laparoscopic promonto fixation is achieved using an anterior and a posterior mesh prosthesis part.

The required material is as follows:

1) Introducers

The introducers comprise helicoidal needles, one having a clockwise configuration and the other one having an anticlockwise configuration (only a needle with a clockwise configuration is shown in FIGS. 7A and 7B).

The angle and the lead of the helix, as well as the angle at the tip (which can be different from the current angle), can be defined by the man skilled in the art, possibly a set of needles with different angles and leads can be proposed to the surgeon.

Optionally, a straight end can be added at the tip of the needle to help the introduction through the wall. If so, said end should be detachable and smooth pointed.

Furthermore, the end of the working tip is provided with means able to catch the prosthesis parts, such as a notch or a hook formation.

Said needles are preferably associated with tunnelisers or canula having a length adapted to their temporary implantation sites (as for the PROLIFT procedure).

2) Two Part Prosthesis Made of Polypropylene Mesh

According to a first alternative, said prosthesis can have a constitution as shown in FIGS. 4A and 5A and as described before.

According to a second alternative, said prosthesis can have a constitution as shown in FIGS. 4B and 5B, namely:
an anterior part having a triangular shape with the tip truncated (shortened by cutting off the top of the end), the length of the triangle being 6 cm. There is one opening in the center just below the upper edge of the mesh and the two arms are 1 cm wide and extend from the upper edge of the mesh on both sides with a length of 8-10 cm with a buttonhole in the end of each arm;
a posterior part having a rectangular shape (for example 5×8 cm), with two arms (for example 4 cm in length) extending from the upper edge of the mesh and two buttonholes in the lower left and right corners of the mesh.

The surgical procedure according to the second embodiment of the invention will now be described in connection with the FIGS. 6 to 14 of the drawings.

The main steps of said procedure are as follows:

1. Preparing a Set-Up as for a Conventional Laparoscopy with (FIG. 6):
a 10 mm trocar for the camera
three 5 mm trocars (the position should be adjusted according to the clinical situation): one suprapubic trocar and two lateral trocars

2. Steps of the Dissection:

2.1 Rectovaginal dissection down to the levator ani

2.2 Identification of uterosacral ligaments

2.3 Subtotal hysterectomy (optional)

2.4 Dissection of vesico-vaginal space

3. Introduction and Placement of the Posterior Mesh (Posterior Prosthesis Part):

3.1 Insert the right introducer through the puborectal muscle from outside to inside and grasp the right arm of the mesh. Pass the mesh through the tissues, rotating the introducer clockwise. The tension will be adjusted later.

3.2 The insertion of the left arm of the mesh is done by reverse movements described in 3.1 and using the left introducer

4. Placement of the Anterior Mesh (Anterior Prosthesis Part):

4.1 The mesh has to be attached low in the vesico-vaginal space. It can be done by the suture (or staples, or glue or trans tissular passage). In any case the vaginal mucosa should not be passed.

4.2 In case of subtotal hysterectomy one suture is passed to close the cervix. This suture is then preferably used to suspend the cervix and to free the assistant. This suture passes in the anterior part (for example through an inbuilt hole) and can be used tu suspend the vesico-uterine peritoneum.

4.3 Fixing of the arms

Two hypotheses have to be considered:

First hypothesis: with only one passage (insertion) of the introducer.

Second hypothesis: with two passages (insertions) of the introducer.

The introducer has to go through the following tissues in the indicated order to achieve the desired effect:

a. Rectovaginal peritoneum

b. Uterosacral-cardinal complex

c. Vesico-uterine fascia (optional)

d. Vesico-uterine peritoneum, OUT-IN

e. The arms of the anterior mesh.

The rotation of 360 degrees of the introducer has to be performed after every passage through these tissues.

If the one passage technique is chosen, the first movement will be to catch the homolateral root of the uterosacral ligament.

If the two passages technique is chosen, the next step will consist of passing the corresponding introducer through the uterosacral ligament until its insertion into the torus uterinus, then grasp the corresponding arm of the mesh as a final step of the preceding manoeuver.

5. Tension Adjustment:

The tension is applied to the right and left arms simultaneously to bring the vaginal vault to a desired position. The excess of the mesh is cut off.

The rationale for the new procedure is to optimize the repair of pelvic organ prolapse by keeping the known benefits of both vaginal and laparoscopic approaches while getting rid of their respective drawbacks.

1. Keeping the Benefits of the Laparoscopic Approach
The dissection is wide and precise due to the excellent intraoperative visibility.
It is possible to visually control that the prosthesis has been put in place where it was intended to be in order to make sure the repair was correctly performed on an anatomic standpoint.
The prosthesis is usually well tolerated and in any case better than when passed through the vagina.
The clinical outcomes of laparoscopic sacro-colpo-pexies are excellent and substantiated by clinical evidence.

2. Getting Rid of the Drawbacks of the Laparoscopic Approach
The lap sacro-colpo-pexy is a long and difficult procedure. This issue is the main reason why the procedure never gained wide acceptance. The invention as described will offer a dramatic simplification of the procedure.
There are wide variations in the way sacro-colpo-pexies are performed all over the world. One of the purpose of the present invention is to offer a route to standardization.
There is a need for performing multiple endoscopic sutures. These sutures are technically difficult for a non-expert surgeon. The procedure as described by the inventors will lead to a significant reduction of the need for endoscopic sutures.
It is often considered that the level for suspension is too high resulting in a sub-optimal repair on an anatomical or functional standpoint. The procedures as described will address this issue by lowering the level of the fixation.
Some urinary symptoms such as de novo urinary incontinence or bladder instability are common complications of the lap approach.

3. Keeping the Benefits of the Vaginal Approach
The prosthesis part(s) is (are) not placed under tension (“tension free” concept).

The fixation of the prosthesis is based on its friction to the surrounding tissues.

The procedure is well described and has become a standard due to its broad diffusion.

4. Getting Rid of the Drawbacks of the Vaginal Approach
The need for opening the vagina is associated with well-known risks such as vaginal erosions and local infections. One of the major benefit of the procedure as described in the present invention is the avoidance of any opening of the vaginal wall.
The prosthesis can also shrink and this could be favoured by the vaginal approach.
The vaginal procedure comprises some steps which are performed in a blind manner, in particular the blind passage of large diameter needles through the tissues. This is associated with a risk of nerve or vessel injury which can result in severe hemorrhage. There is no need for such passages in the procedure as described in the present invention.

1. A prosthesis to be implanted in a body for repairing or treating pelvic organ prolapses in a female patient, said one piece prosthesis made of synthetic, biological or mixed mesh like fabric, said prosthesis having roughly a human shape like outline with two arm portions (A), two leg portions (P), a trunc portion (p) and a head portion (a), the head and trunc portions forming together substantially a rectangular shape divided into an interior part and a posterior part by a hypothetical medium line (M) crossing the trunc portion under its junctions with the arm portions; the posterior part being provided with two separated holes (o) situated near the medium line, the anterior part (a) being adapted to be placed into the vesico-vaginal space and attached low in said space, the posterior part (p) being adapted to be placed into the rectovaginal space, the leg portions being adapted to be passed through and anchored into the right and left puborectal muscles and the arm portions being adapted to be passed through the holes of the posterior part and embedded within and/or attached to the uterosacral ligaments.

2. A prosthesis to be implanted in the body of a female patient for repairing or treating pelvic organ prolapses, said prosthesis being formed of two parts made of synthetic, biological or mixed mesh like fabric,
wherein a first or anterior part comprises a main rectangular shaped body (MBA) provided with two arm like anterior strap portions (a) extending with a diverging angle from the corners adjacent a small side of the main body, said main body having a truncated shape at its other small side and a hole (o) near the edge of the small side between the junctions with the arms (a),
wherein a second or posterior part comprises a main rectangular shaped body (MBP) provided with two arm like posterior strap portions (pa) extending with a diverging angle and a slightly curved shape from the corners adjacent a small side of the main body, said main body having two separated holes (h) next to its other opposite small side,
the main body of the anterior part being adapted to be placed into the vesico-vaginal space and attached low in said space, with the corresponding anterior strap portions adapted to be passed, once or twice, through the holes of the posterior part and attached to the uterosacral ligaments, the main body of the posterior part being adapted to be placed into the rectovaginal space, with the corresponding posterior strap portions adapted to pass through and being anchored into the right and left puborectal muscles.

3. A prosthesis according to claim 1, characterised in that the mesh fabric consists of a bio-compatible material fabric and in that the dimensions of the prosthesis constituting parts or portions are adapted to the size of the patient's anatomy.

4. A procedure for repairing or treating pelvic organ prolapses in a female patient using a synthetic, biological or mixed implantable prosthesis, comprising the steps of:
a) setting-up the patient as for a conventional laparoscopy;
b) performing a rectovaginal dissection down to the levator-ani;
c) performing a dissection of the vesico-vaginal space, preferably up to the bladder trigone;
d) providing a prosthesis made of mesh like fabric and formed by central anterior and posterior portions and lateral arm and possible leg portions, preferably a prosthesis according to claim 1, and inserting said prosthesis through the dissected area so as to place the posterior part into the rectovaginal space;
e) anchoring the posterior leg portions into the perineum or leaving the posterior part free in the rectovaginal space;
f) folding down the anterior part into the dissected vesico-vaginal space;
g) passing the anterior arm portions through the holes of the posterior part and anchoring said arm portions into the uterosacral ligaments or passing said anterior arm portions under the peritoneum or along the uterosacral ligament in order to restore a physiological suspension of the uterus, the cervix or the vaginal apex in case of post hysterectomy prolapse;
h) adjusting the position of the concerned organs by applying tension to the arm and leg portions, the suspension being achieved by the friction of the mesh in the native tissues;
i) closing up the patient.

5. A procedure according to claim 4, wherein a subtotal hysterectomy is performed after step c) and the uterine cervix is closed and a needle suspension to the anterior abdominal wall is performed after step d).

6. A procedure for repairing or treating pelvic organ prolapses in a female patient using a synthetic, biological or mixed implantable prosthesis, comprising the steps of:
a) setting-up the patient as for a conventional laparoscopy;
b) performing a rectovaginal dissection down to the levator-ani;
c) performing a dissection of the vesico-vaginal space, preferably up to the bladder trigone;
d) providing a two part prosthesis comprised of an anterior part having a central body and two arm like strap portions and a posterior part having a central body and two arm like strap portions, preferably a prosthesis according to claim 2;
e) introducing and placing the posterior part into the rectovaginal space and pressing through and anchoring the posterior strap portions into the right and left puborectal muscles;
f) introducing and placing the anterior part into the vesico-vaginal space;
g) passing the anterior strap portions, once or twice, through the holes of the posterior part and attaching them to the uterosacral ligaments;
h) adjusting the position of the concerned organs by applying tension to the strap portions;
i) closing up the patient.

7. A procedure according to claim 6, wherein a subtotal hysterectomy is performed after step c) and a suture is passed to close the cervix, said suture passing also through an inbuild hole of the anterior part.

8. A procedure according to claim 4, wherein the passing, placing and attaching of the arm and/or leg portions of the prosthesis is performed by grasping and then pulling said portions by their respective free ends by means of an introducer comprising a specifically shaped needle (see figure), possibly cooperating with a corresponding canula.

9. A procedure according to claim 8, wherein the anchoring of the posterior leg or strap portions is performed by passing the introducer through the puborectal muscles within two lateral incisions made in the perineum.

10. A procedure according to claim 8, wherein the attachment of the anterior arm portions is performed by passing the introducer through incisions of the perineum and basting the needle under the peritoneum until the root of the uterosacral ligament is transfixed, before grasping the free end of the concerned anterior arm portion.

11. A procedure according to claim 8, wherein the attachment of the anterior strap portions to the uterosacral ligament is performed by passing the introducer successively through the following tissues: recto-vaginal peritoneum, uterosacral-cardinal complex, possibly the vesico-uterine fascia, in and out the vesico-uterine peritoneum, before grasping the free end of the concerned anterior strap portion.

12. A procedure according to claim 4, wherein the free edge of the anterior part of the prosthesis is attached low in the vesico-vaginal space.

13. A procedure according to claim 4, wherein the posterior part of the prosthesis is without leg portions or posterior strap portions, the posterior part of the prosthesis being free or suspended to the muscle by an adapted means.

14. A prosthesis according to claim 2, characterised in that the mesh fabric consists of a bio-compatible material fabric and in that the dimensions of the prosthesis constituting parts or portions are adapted to the size of the patient's anatomy.

15. A procedure according to claim 5, wherein the passing, placing and attaching of the arm and/or leg portions of the prosthesis is performed by grasping and then pulling said portions by their respective free ends by means of an introducer comprising a specifically shaped needle (see figure), possibly cooperating with a corresponding canula.

16. A procedure according to claim 6, wherein the passing, placing and attaching of the arm and/or leg portions of the prosthesis is performed by grasping and then pulling said portions by their respective free ends by means of an introducer comprising a specifically shaped needle (see figure), possibly cooperating with a corresponding canula.

17. A procedure according to claim 7, wherein the passing, placing and attaching of the arm and/or leg portions of the prosthesis is performed by grasping and then pulling said portions by their respective free ends by means of an introducer comprising a specifically shaped needle (see figure), possibly cooperating with a corresponding canula.

18. A procedure according to claim 5, wherein the free edge of the anterior part of the prosthesis is attached low in the vesico-vaginal space.

19. A procedure according to claim 6, wherein the free edge of the anterior part of the prosthesis is attached low in the vesico-vaginal space.

20. A procedure according to claim 7, wherein the free edge of the anterior part of the prosthesis is attached low in the vesico-vaginal space.
